(19) United States
(12) Plant Patent Application Publication
(10) Pub. No.: US 2019/0297762 P1
(43) Pub. Date: Sep. 26, 2019

(54) Rhododendron L. Plant Named 'Sweetheart'

(50) ;

(71) Applicant: Akatsuka Garden Co., Ltd., Tsu City (JP)

(72) Inventor: Yukio Kurabayashi, Tsu City (JP)

(21) Appl. No.: 15/932,620

(22) Filed: Mar. 23, 2018

(51) Int. Cl. A01H 6/36

(52) U.S. Cl. PLT/238

(57) ABSTRACT

‘Sweetheart’ is a new variety of rhododendron. This variety was produced by the hybridization of the ‘Coral Moon’ variety with the ‘Atlas’ variety in 2011. The plant may be used, e.g., as a flowering plant for outdoor cultivation, or as a potted plant. ‘Sweetheart’ is visibly appealing, and produces flowers comprising a corolla that comprises bright red-purple and light purple-pink colors.

Plant name and variety denomination: Latin name of the genus and species of the plant claimed: Rhododendron L. The claimed plant is a hybridization of different species. The parental lines of the claimed plant resulted from the hybridization of different species. Common names of the claimed plant's species: Rhododendron, Azalea, Azaleodendron. Variety denomination: ‘Sweetheart’.

### BACKGROUND OF THE INVENTION

Rhododendron is a genus of woody plants in the Ericaceae family, either evergreen or deciduous, and found mainly in Asia, although it is also widespread throughout the highlands of the Appalachian Mountains of North America. Most species have brightly colored flowers that bloom from late winter through to early summer.

Both species and hybrid rhododendrons (including azaleas) are used extensively as ornamental plants in landscaping in many parts of the world, including both temperate and subtemperate regions. Many species and cultivars are grown commercially for the nursery trade.

Rhododendrons are often valued in landscaping for their structure, size, flowers, and the fact that many of them are evergreen. Azaleas are frequently used around foundations and occasionally as hedges, and many larger-leafed rhododendrons lend themselves well to more informal plantings and woodland gardens, or as specimen plants.

### BRIEF SUMMARY OF THE INVENTION

‘Sweetheart’ is a new variety of rhododendron (more specifically azalea, still more specifically azaleodendron) bred by pollinating variety ‘Coral Moon’ with pollen from variety ‘Atlas’ in 2011. The plant may be used, e.g., as a flowering plant for outdoor cultivation or as a potted plant.

‘Sweetheart’ is visibly appealing, and produces flowers comprising a corolla that has spots and bright red-purple and light purple-pink colors.

‘Coral Moon’ rhododendron (unpatented) is a rhododendron variety that originated in Japan in 2003. ‘Coral Moon’ was produced by the hybridization of the ‘Rouge Magic’ (unpatented) variety with the ‘Wedding Bouquet’ (unpatented) variety. This variety has a small tree size in the shape of a dome. The flowers of this variety have a small diameter with a bright pinkish-red color. There are about 15 pieces of blossom flower in the shape of a small dome.

‘Atlas’ rhododendron (unpatented) is a rhododendron variety that originated in Japan in 2005. ‘Atlas’ was produced by the hybridization of the ‘Helios’ variety with the ‘Blue Eye’ variety. This variety has a medium tree size and spreading branches. The flowers of this variety have a medium diameter and a pale red color. There are about 15 pieces of flower blossoms in the shape of a pyramid.

‘Sweetheart’ was asexually reproduced by tissue culture in Japan at Kamiogoso2080, Anocho Toshima, Tsu-shi, Mie, 514-2322. Specifically, propagation was carried out by taking a growing point from a lateral bud of a sprout and cultivating the growing point, without use of callus. Plants propagated from a ‘Sweetheart’ plant by asexual or vegetative means are uniform and stable.

### BRIEF DESCRIPTION OF THE DRAWINGS

FIG. 1 is a photograph of a ‘Sweetheart’ plant body.

FIG. 2 is a photograph depicting the full view of young ‘Sweetheart’ plants being cultivated in a divided tray.

FIG. 3 is a photograph showing an enlarged view of a ‘Sweetheart’ flower.

FIG. 4 is a photograph of a dissected ‘Sweetheart’ flower.

FIG. 5 is a photograph of flowers of comparative variety ‘Mrs. Fujii’.

FIGS. 6A-D are photographs showing measurements of various different parts of ‘Sweetheart’ plants. The units shown are centimeters.

FIG. 7 is a photograph of the plant body (including flowers) of parental variety ‘Atlas’.

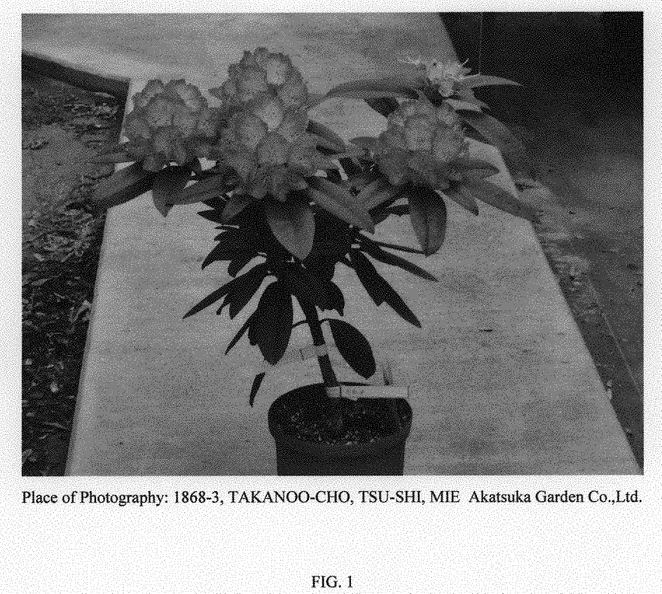

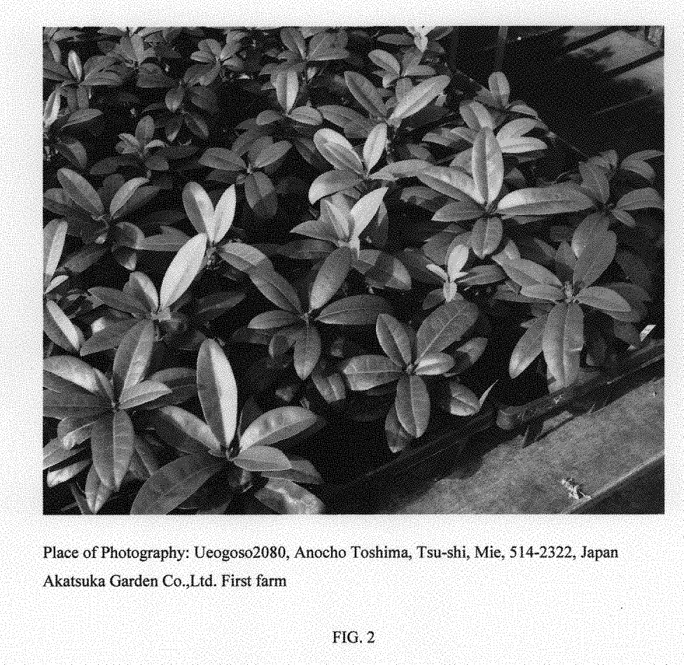

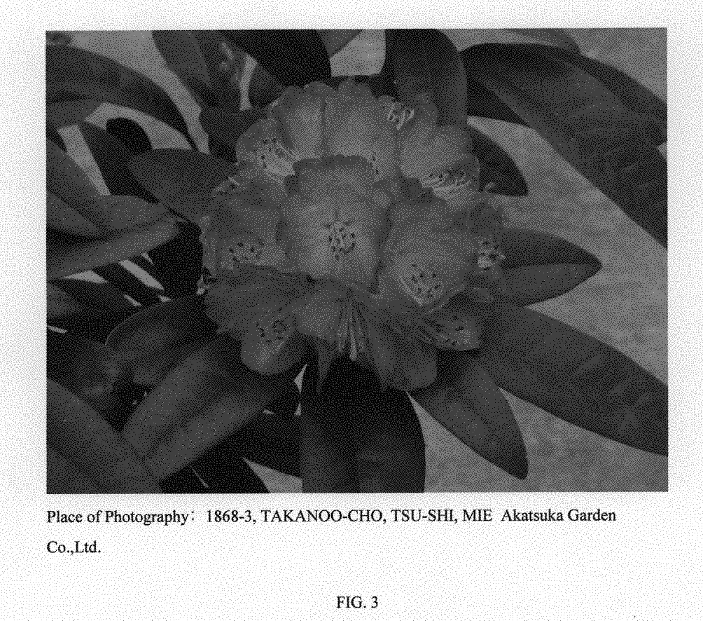

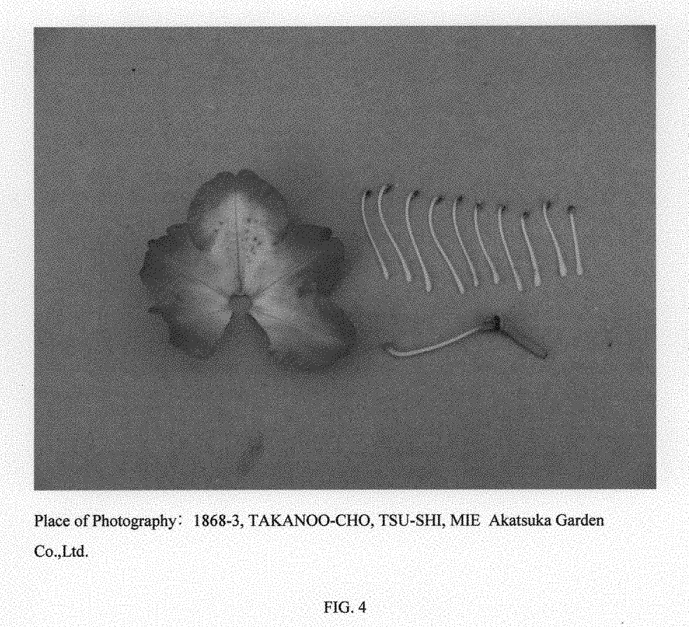

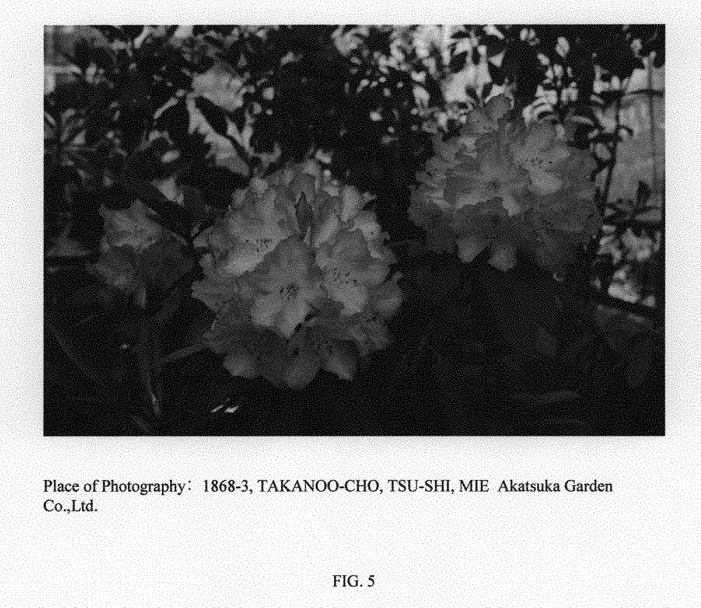

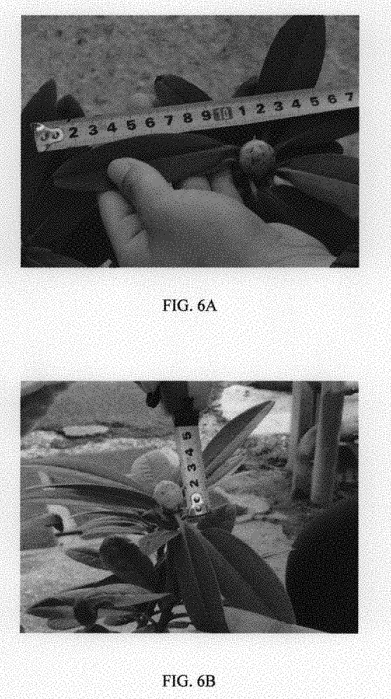

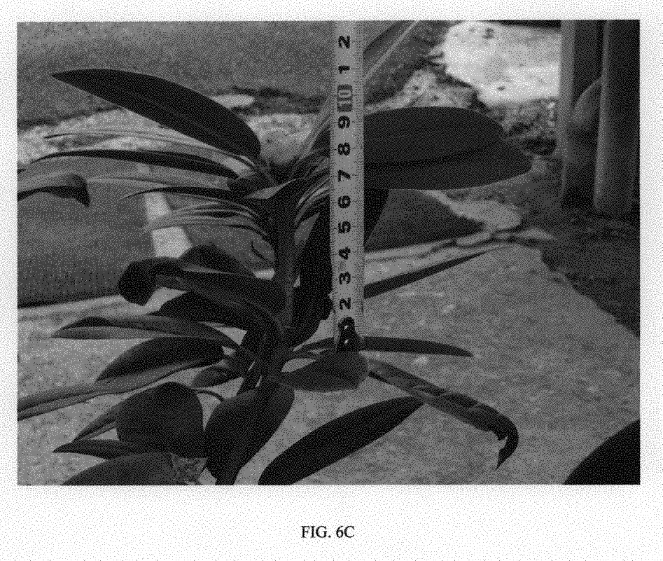

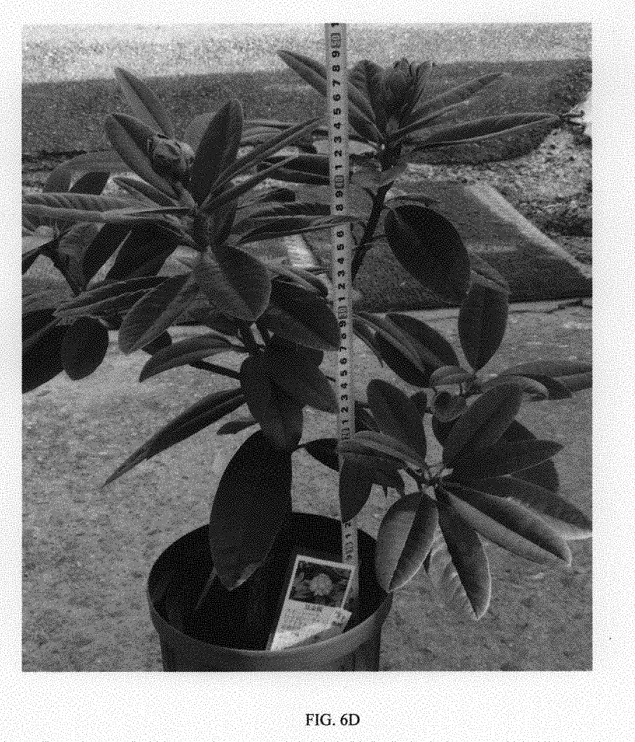

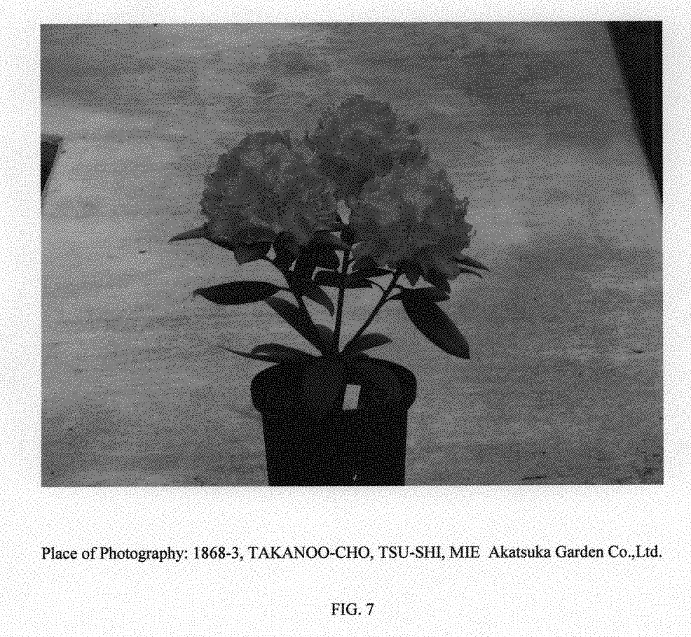

### DETAILED BOTANICAL DESCRIPTION OF THE PLANT

Variety: ‘Sweetheart’

Species of the plant claimed: Rhododendron L. The claimed plant is a hybridization of different species. The parental lines of the claimed plant resulted from the hybridization of different species.

Common names of the claimed plant's species include: Rhododendron, Azalea, Azaleodendron

Characteristics of ‘Sweetheart’ provided herein were observed when the plant was cultivated in the open with rain cover at Akatsuka Garden Co., Ltd., “first farm” having an address at Ueogoso2080, Anocho Toshima, Tsu-shi, Mie, 514-2322, Japan. Descriptive terms used in Tables 1, 2, and 3 herein are consistent with the Japanese Examination Standard for Rhododendron/Azalea (Rhododendron L.), TG/42/6 1995-10-20, pages 1-17, available at www.hinshu2.maff.go.jp/info/sinsakijun/kijun/1543.pdf, the entire contents of which are incorporated herein by reference.

Properties and characteristics of ‘Sweetheart’ are described in Table 1.

TABLE 1
Properties and characteristics of ‘Sweetheart’

| | | | Class Value |
|---|---|---|---|
| | | | (Numeric |
| Charact- | | | Data) of |
| eristic | | | ‘Sweet- |
| No. | Description | State of Expression | heart’** |
| 1 | Plant: persistence of | evergreen | 3 |
| | leaves | | |
| 24 | Flower: shape | open funnel-shaped | 2 |
| 25 | Flower: diameter | medium | 5 |
| 30 | Corolla lobe: number of | two | 2 |
| | colors on upper side | | |
| | (excluding markings of | | |
| | the throat) | | |
| 31 | Corolla lobe: main color | Japan color standard | Not |
| | on upper side | for horticultural plants | Applicable |
| | | (JHS) Color Chart No. | |
| | | 9506 (bright red- | |
| | | purple) | |
| 32 | Corolla lobe: secondary | JHS Color Chart No. | Not |
| | color on upper side | 9502 (light purple- | Applicable |
| | | pink) | |
| 35 | Corolla lobe: | medium | 5 |
| | conspicuousness of | | |
| | markings of the throat | | |
| 36 | Corolla lobe: type of | spots not touching | 1 |
| | markings | each other | |
| 42 | Time of beginning of | late* | 7 |
| | flowering | | |

*Early April to mid-April, e.g., April 5^{th}, when grown at Akatsuka Garden Co., Ltd., “first farm” having an address at Ueogoso2080, Anocho Toshima, Tsu-shi, Mie, 514-2322, Japan.
**Numerical Values in this column correspond to the “state” in the Table of Characteristics of the Japanese Examination Standard for Rhododendron/Azalea (Rhododendron L.), TG/42/6 1995-10-20, pages 1-17, available at www.hinshu2.maff.go.jp/info/sinsakijun/kijun/1543.pdf.

When grown in Tsu-shi, Mie, 514-2322, Japan (or in an areas with a similar latitude and climate) ‘Sweetheart’ should be planted between early October to late March. In this region, flowering time is from early April to mid-April. Locations suitable for the growth of ‘Sweetheart’ included mesic areas with good drainage and a soil pH of 5.0 to 5.5.

A variety that is similar to ‘Sweetheart’ is ‘Mrs. Fuji’. Characteristics of ‘Sweetheart’ and ‘Mrs. Fuji’ are compared in Table 2. Flowers of ‘Mrs. Fuji’ are shown in FIG. 5.

TABLE 2
Distinguishable Characteristics between ‘Sweetheart’ and ‘Mrs. Fujii’

| Denom- | | State of | |
|---|---|---|---|
| ination | | Expression in | State of |
| of Similar | Description of | Similar | Expression in |
| Variety | Characteristics | Variety | Candidate Variety |
| Mrs. Fujii | Corola lobe: main color | 9204 (vivid | 9506 (bright |
| | on upper side | purple-pink) | red-purple) |

A comparison between features of ‘Sweetheart’ and its parental varieties is provided in Table 3.

TABLE 3
Description of Physical Differences between Parental Plants

| | Characteristic of | | |
|---|---|---|---|
| Variety | flower | Shape of tree, etc. | Origin |
| Clained Variety: | Medium diameter with | Medium size of tree | First hybridized in 2011. |
| ‘Sweetheart’ | edge color, red-pink | having spreading | Flowering first observed |
| | and white in the | branch | in 2014. |
| | bottom. 25 pieces of | | |
| | blossom flower in the | | |
| | shape of pyramid | | |
| Mother Variety: | Small diameter flowers, | Small size of tree in the | Original variety obtained |
| ‘Coral Moon’ | bright pinkish-red. 15 | shape of a dome. | by hybridization of |
| | pieces of blossom | | ‘Rouge Magic’ variety |
| | flower in the shape of | | with ‘Wedding Bouquet’ |
| | small dome. | | variety in 2003. |
| Father Variety: | Medium diameter | Medium size of tree | Original variety obtained |
| ‘Atlas’ | flower, pale red. | having spreading | by hybridization of |
| | Around 15 pieces of | branch. | ‘Helios’ variety with |
| | blossom flower in the | | ‘Blue Eye’ variety in |
| | shape of pyramid | | 2005. |

It will be understood that the average size of the plant may vary with location, season, nutrition, irrigation, etc.

What is claimed is:

1. A new and distinct Rhododendron L. plant named ‘Sweetheart’ as illustrated and described.
